SEQUENCE LISTING (3 sequences)

SEQ ID NO: 1 - DNA, 22 bp, Artificial Sequence
  Synthetic Construct
tgactgtgaacgttcgagatga

SEQ ID NO: 2 - DNA, 11 bp, Artificial Sequence
  Synthetic Construct
  misc_feature 3, 7, 11: 2′-deoxy-7-deazaguanosine
tcgaacgttcg

SEQ ID NO: 3 - DNA, 10 bp, Artificial Sequence
  Synthetic Construct
aacgttcgag

(19) United States
(12) Patent Application Publication
(10) Pub. No.: US 2023/0072809 A1
(43) Pub. Date: Mar. 9, 2023

(54) ACTIVE BOOSTER IMMUNIZATION AGAINST TETANUS, DIPHTHERIA AND PERTUSSIS

(71) Applicants: Dynavax Technologies Corporation, Emeryville, CA (US); Serum Institute of India Private Limited, Pune (IN)

(72) Inventors: John D. CAMPBELL, San Francisco, CA (US); Robert L. COFFMAN, Portola Valley, CA (US); Randall N. HYER, Gwynedd Valley, PA (US); Robert S. JANSSEN, Palm Springs, CA (US); David NOVACK, Oakland, CA (US); Martin GOHLKE, Berkeley, CA (US); Maureen URBAN, El Cerrito, CA (US); Sunil GAIROLA, Pune (IN); Umesh SHALLIGRAM, Pune (IN); Manish GAUTUM, Pune (IN); Harish RAO, Pune (IN)

(73) Assignees: Dynavax Technologies Corporation, Emeryville, CA (US); Serum Institute of India Private Limited, Pune (IN)

(21) Appl. No.: 17/910,332

(22) PCT Filed: Mar. 9, 2021
(86) PCT No.: PCT/US2021/021532

§ 371 Date: Sep. 8, 2022

(30) Foreign Application Priority Data
Mar. 9, 2020 (IN) 202011010025
Dec. 19, 2020 (IN) 202011055399

(51) Int. Cl. A61K 39/02; A61K 39/08; A61K 39/05; A61K 39/13; A61P 31/04

(52) U.S. Cl. CPC A61K 39/099; A61K 39/08; A61K 39/05; A61K 39/13; A61P 31/04; A61K 2039/70

(57) ABSTRACT

The present disclosure relates to immunogenic compositions comprising tetanus, diphtheria, and acellular pertussis (Tdap) antigens, and a toll-like receptor 9 (TLR9) agonist, such as oligonucleotide comprising an unmethylated cytidine-phospho-guanosine (CpG) motif. The immunogenic compositions may further comprise an aluminum salt adjuvant to which the Tdap antigens are adsorbed. The immunogenic compositions are suitable for active booster immunization against tetanus, diphtheria, and pertussis in an individual in need thereof.

### CROSS-REFERENCE TO RELATED APPLICATIONS

This application claims priority to and the benefit of Indian Provisional Application No. 202011010025, filed Mar. 9, 2020, and Indian Provisional Application No. 202011055399, filed Dec. 19, 2020, the disclosure of each of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 377882006842SEQLIST.TXT, date recorded: Mar. 8, 2021, size: 2 KB).

### FIELD

The present disclosure relates to immunogenic compositions comprising tetanus, diphtheria, and acellular pertussis (Tdap) antigens, and a toll-like receptor 9 (TLR9) agonist, such as oligonucleotide comprising an unmethylated cytidine-phospho-guanosine (CpG) motif. The immunogenic compositions may further comprise an aluminum salt adjuvant to which the Tdap antigens are adsorbed. The immunogenic compositions are suitable for active booster immunization against tetanus, diphtheria, and pertussis in an individual in need thereof.

### BACKGROUND

Pertussis is a highly infectious disease and remains an important worldwide public health problem, even in countries with sustained high vaccination coverage. Despite established infant immunization programs, pertussis continues to circulate, predominantly due to waning immunity beyond childhood and disease transmission from adolescents and adults to vulnerable infants. Safety concerns about the whole-cell pertussis component of combined diphtheria, tetanus and whole-cell pertussis (DTwP) vaccines prompted the switch to combined diphtheria, tetanus and acellular pertussis (DTaP) vaccines in the 1990s. However, declining immunity to pertussis in individuals completing a pediatric DTaP vaccine regimen has resulted in an increase in incidence of pertussis since the mid-2000s (Liang et al., MMWR Recomm Rep, 67(2):1-43, 2018). Similarly, tetanus and diphtheria antitoxin levels fall over time in individuals completing a pediatric DTaP vaccine regimen.

The Advisory Committee on Immunization Practices (ACIP) now recommends administration of a single booster dose of a tetanus toxoid, reduced diphtheria toxoid, and acellular pertussis (Tdap) vaccine to individuals over the age of 10, and a booster dose of a either a Tdap vaccine or a tetanus and diphtheria (Td) vaccine at 10 year intervals or as needed for wound management (Liang et al., supra, 2018). Clinical trials have shown that two doses of Tdap are safe and immunogenic, but that anti-pertussis antibody titers rapidly decline, generally resulting in loss of protection against pertussis infection within two to three years.

Thus, improved booster vaccines are needed to provide higher titer and longer lived antibody responses against pertussis antigens to help address the waning immunity observed as a consequence of the switch from whole-cell to acellular pertussis antigens in current vaccine regimens Improved Tdap booster vaccines are needed, which have a satisfactory safety profile and are not inferior to current vaccines with regard to tetanus and diphtheria toxoid immunogenicity.

### SUMMARY

The present disclosure relates to immunogenic compositions comprising tetanus, diphtheria, and acellular pertussis (Tdap) antigens, and a toll-like receptor 9 (TLR9) agonist, such as oligonucleotide comprising an unmethylated cytidine-phospho-guanosine (CpG) motif. The immunogenic compositions may further comprise an aluminum salt adjuvant to which the Tdap antigens are adsorbed. The immunogenic compositions are suitable for active booster immunization against tetanus, diphtheria, and pertussis in an individual in need thereof.

### BRIEF DESCRIPTION OF THE DRAWINGS

FIG. 1A-C depicts the levels of cytokine secretion by spleen cells in vitro after restimulation with Tdap antigens. Levels of IFN-γ (FIG. 1A), IL-5 (FIG. 1B), and IL-17 (FIG. 1C) are shown.

FIG. 2A-B shows that inclusion of CpG 1018 in Tdap vaccines results in a substantial reduction in B. pertussis burden in the lungs and trachea three days after intranasal challenge of vaccinated mice.

FIG. 3A-C shows that inclusion of CpG 1018 in Tdap vaccines results in an increase in serum antibody titers prior to intranasal challenge. FIG. 3A show B. pertussis-reactive titers, FIG. 3B show PT reactive titers, and FIG. 3C show FHA-reactive titers.

### GENERAL TECHNIQUES AND DEFINITIONS

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 μg of CpG 1018 refers to 2700 μg to 3300 μg of CpG 1018).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG”, “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation or expansion of lymphocyte populations, such as NK cells, CD4+ T lymphocytes, CD8+ T lymphocytes, B lymphocytes, and the like. Preferably, the CpG oligonucleotide preferentially activates a Th1-type response.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient antigen and TLR9 agonist to stimulate an immune response (preferably a seroprotective level of antibody to the antigen).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection.

The term “vaccination” as used herein refers to the introduction of vaccine into a body of a mammalian subject.

“Adjuvant” refers to a substance which, when added to a composition comprising an antigen, nonspecifically enhances or potentiates an immune response to the antigen in the mammalian recipient upon exposure.

### DETAILED DESCRIPTION

The present disclosure relates to immunogenic compositions comprising tetanus, diphtheria, and acellular pertussis (Tdap) antigens, and a toll-like receptor 9 (TLR9) agonist, such as oligonucleotide comprising an unmethylated cytidine-phospho-guanosine (CpG) motif. The immunogenic compositions may further comprise an aluminum salt adjuvant to which the Tdap antigens are adsorbed. The immunogenic compositions are suitable for active booster immunization against tetanus, diphtheria, and pertussis in an individual in need thereof.

### I. Immunogenic Compositions and Kits

The present disclosure relates to immunogenic compositions for active booster immunization against tetanus, diphtheria, and pertussis, comprising tetanus, diphtheria, and acellular pertussis (Tdap) antigens, and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, the Tdap antigens comprise a tetanus toxoid (TT), a diphtheria toxoid (DT), a pertussis toxoid (PT), a pertussis filamentous haemagglutinin (FHA), and pertussis pertactin (PRN), and the Tdap antigens and the oligonucleotide are present in the immunogenic composition in amounts effective to stimulate an immune response against the Tdap antigens in a human subject. In some embodiments, the immunogenic compositions further comprise an aluminum salt adjuvant to which the Tdap antigens are adsorbed.

A. Toll-Like Receptor 9 (TLR9) Agonists

Toll-like receptors (TLRs) are expressed on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, Calif.) have identified a 22-mer phosphorothioate linked oligodeoxynucleotide, CpG 1018, which contains specific sequences that can substantially enhance the immune response to co-administered antigens across species (Campbell, Methods Mol Biol, 1494:15-27, 2017). CpG 1018 (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018 is a CpG-B ODN that is active in mice, rabbits, dogs, baboons, cynomolgus monkeys, and humans. Thus in some preferred embodiments, the TLR9 agonist is an oligonucleotide comprising the sequence of SEQ ID NO:1.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which is typically a 0.5 ml dose, may comprises from about 375 μg to about 6000 μg of the CpG oligonucleotide, preferably from about 750 μg to about 3000 μg of the CpG oligonucleotide. In some embodiments, a 0.5 ml dose of the immunogenic composition comprises greater than about 250, 500, 750, 1000, or 1250 μg of the CpG oligonucleotide, and less than about 6000, 5000, 4000, 3000, or 2000 μg of the CpG oligonucleotide. In some embodiments, a 0.5 ml dose of the immunogenic composition comprises about 375, 750, 1500, 3000 or 6000 μg of the CpG oligonucleotide. In some embodiments, a 0.5 ml dose of the immunogenic composition comprises about 750 μg of the CpG oligonucleotide. In some embodiments, a 0.5 ml dose of the immunogenic composition comprises about 1500 μg of the CpG oligonucleotide. In some embodiments, a 0.5 ml dose of the immunogenic composition comprises about 3000 μg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

B. Tetanus, Diphtheria, and Pertussis Antigens

Tetanus is an acute, often fatal disease, characterized by neuromuscular dysfunction caused by a neurotoxin produced by the bacterium Clostridium tetani. Tetanus is contracted through exposure to spores of the bacterium, which are commonly found in soil, dust and manure Immunity to the tetanus neurotoxin is rarely acquired naturally, but protection from disease can be conferred by of administration of a tetanus toxoid (TT)-containing vaccine (Liang et al., MMWR Recomm Rep, 67(2):1-43, 2018). A serum tetanus antitoxin level of at least 0.01 IU/ml as measured in a neutralization assay is considered protective, as is a serum tetanus antitoxin level of at least 0.1 IU/ml as measured by enzyme linked immunosorbent assay (ELISA). The immunogenic compositions of the present disclosure comprise TT produced by chemical detoxification of tetanus toxin produced by C. tetani. In some embodiments, a unit dose (e.g., about 0.5 ml) of the immunogenic composition comprises from about 5-10 limit of flocculation unit (Lf) TT, preferably about 5.0 Lf TT.

Diphtheria is an acute disease caused by toxigenic strains of the bacterium Corynebacterium diphtheriae. Diphtheria is contracted primarily by direct contact with or from inhalation of aerosolized bacteria from C. diphtheriae-infected individuals. Disease is characterized by sore throat and fever, which in severe cases can lead to airway obstruction. Additionally, systemic spread of the diphtheria toxin can cause cardiac and neurologic complications. Diphtheria infection may not confer protective immunity. However, protection from disease can be conferred with a diphtheria toxoid (DT) containing vaccine (Liang et al., MMWR Recomm Rep, 67(2):1-43, 2018). A serum diphtheria antitoxin level of at least 0.01 IU/ml as measured in a neutralization assay may provide some protection, while a serum diphtheria antitoxin level of at least 0.1 IU/ml as measured by ELISA is considered protective, and a diphtheria antitoxin level of at least 1.0 IU/ml is associated with long-term protection. The immunogenic compositions of the present disclosure comprise DT produced by chemical detoxification of diphtheria toxin produced by C. diphtheriae. In some embodiments, a unit dose (e.g., about 0.5 ml) of the immunogenic composition comprises a reduced amount of DT of from 2.0-2.5 Lf DT, preferably about 2.5 Lf DT.

Pertussis, also known as whooping cough, is an acute respiratory disease caused by the bacterium Bordetella pertussis. Pertussis is a highly contagious disease spread through inhalation of aerosolized bacterium from B. pertussis-infected individuals. Disease severity is inversely related to the age of the infected subject, with infants and young toddlers (e.g., birth-2 years old) being at greatest risk of severe symptoms leading to hospitalizations and death. Introduction of pediatric vaccine regimens have decreased the incidence of pertussis, and a correlation between the presence of anti-pertussis antibodies and protection from disease has been observed in vaccine efficacy studies (Liang et al., MMWR Recomm Rep, 67(2):1-43, 2018). However, serologic or laboratory correlates of protection against pertussis have still not been defined. Accordingly, efficacy of new vaccines is based on their safety profile and non-inferior immunogenicity to existing Federal Drug Administration (FDA)-approved vaccines using similarly-manufactured antigens. The immunogenic compositions of the present disclosure comprise several B. pertussis antigens, namely pertussis toxoid (PT), pertussis filamentous haemagglutinin (FHA), and pertussis pertactin (PRN). The immunogenic compositions may further comprise one or more additional pertussis antigens, such as one or more of pertussis fimbriae (FIM) type 2, pertussis FIM type 3, and pertussis adenylate cyclase (AC). In some embodiments, a unit dose (e.g., about 0.5 ml) of the immunogenic composition comprises a reduced amount of one or more pertussis antigens, such as about 2.5-8.0 μg PT, about 5.0-8.0 μg FHA, and about 2.5-3.0 μg PRN, preferably about 8 μg PT, about 8 μg FHA, and about 2.5 μg PRN.

C. Additional Components

The immunogenic compositions of the present disclosure may comprise one or more additional components, such as one or more excipients, another adjuvant, and/or additional antigens.

1. Excipients

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

2. Additional Adjuvants

Adjuvants are known in the art and include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the Tdap antigens are adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide. In some embodiments, a unit dose (e.g., about 0.5 ml) of the immunogenic composition comprises from about 0.25 to about 0.50 mg Al³⁺, preferably from about 0.30 to about 0.40 mg Al³⁺.

In other embodiments, the immunogenic composition further comprises an additional adjuvant. Additional suitable adjuvants include, but are not limited to, squalene-in-water emulsion (e.g., MF59 or AS03), TLR3 agonists (e.g., poly-IC or poly-ICLC), TLR4 agonists (e.g., bacterial lipopolysaccharide derivatives such monophosphoryl lipid A (MPL), and/or a saponin such as Quil A or QS-21, as in AS01 or AS02), TLR5 agonists (bacterial flagellin), and TLR7 and/or TLR8 agonists (imidazoquinoline derivatives such as imiquimod, and resiquimod)(Coffman et al., Immunity, 33:492-503, 2010). In some embodiments, the additional adjuvant comprises MPL and alum (e.g., AS04). For veterinary use and for production of antibodies in non-human animals, mitogenic components of Freund's adjuvant (both complete and incomplete) can be used.

3. Additional Antigens

The immunogenic compositions of the present disclosure may further comprise at least one additional antigen, such as an antigen from another human disease-causing pathogen. In some embodiments, the pathogen is selected from, but not limited to, one or more of poliovirus, hepatitis B virus, and Haemophilus influenzae Type B (HiB). In some embodiments, the at least one additional antigen comprises an inactivated poliovirus, which may comprise one or more of a type 1 virus, a type 2 virus, and a type 3 virus. In some embodiments, the at least one additional antigen comprises a hepatitis B virus surface antigen. In some embodiments, the at least one additional antigen comprises a pneumococcal antigen, and/or a meningococcal antigen. In some embodiments, the at least one additional antigen comprises Haemophilus b capsular polysaccharide (polyribosyl-ribitol-phosphate). In some embodiments, the Haemophilus b capsular polysaccharide is conjugated to the tetanus toxoid or to a meningococcal protein.

D. Kits

The present disclosure also provides kits comprising: i) an immunogenic composition comprising tetanus, diphtheria, and acellular pertussis (Tdap) antigens, and a toll-like receptor 9 (TLR9) agonist, such as a CpG oligonucleotide; and ii) a set of instructions for administration of the immunogenic composition to stimulate an immune response against Tdap antigens in a human subject. Additionally, the present disclosure provides kits comprising: i) a first composition comprising tetanus, diphtheria, and acellular pertussis (Tdap) antigens and an aluminum salt adjuvant to which the Tdap antigens are adsorbed; ii) a second composition comprising a TLR9 agonist, such as a CpG oligonucleotide; iii) instructions for mixing the first composition with the second composition to prepare an immunogenic composition; and optionally iv) a further set of instructions for administration of the immunogenic composition to stimulate an immune response against Tdap antigens in a human subject. In some embodiments, the CpG oligonucleotide comprises the sequence 5′-AACGTTCG-3′. In some embodiments, the CpG oligonucleotide comprises the sequence 5′-AACGTTCGAG-3′ (SEQ ID NO:3). In some preferred embodiments, the CpG oligonucleotide comprises the sequence of 5′-TGACTGTGAA CGTTCGAGAT GA-3′ (SEQ ID NO:1).

The kits may comprise an immunogenic composition packaged appropriately. For example, if the immunogenic composition is a freeze-dried power, a vial with a resilient stopper is normally used so that the powder may be easily resuspended by injecting fluid (e.g., sterile water, saline, etc.) through the resilient stopper. In some embodiments, the kits comprise a device for administration (e.g., syringe and needle). The instructions relating to the use of the immunogenic composition generally include information as to dosage, schedule and route of administration for the intended methods of use. In some embodiments, the immunogenic compositions are for active booster immunization against tetanus, diphtheria, and pertussis.

### II. Methods of Use

The present disclosure relates to methods for active booster immunization against tetanus, diphtheria, and pertussis, comprising: administering to a human subject an immunogenic composition comprising tetanus, diphtheria, and acellular pertussis (Tdap) antigens, and a Toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, the human subject is three years of age or older, the Tdap antigens comprise a tetanus toxoid (TT), a diphtheria toxoid (DT), a pertussis toxoid (PT), a pertussis filamentous haemagglutinin (FHA), and pertussis pertactin (PRN), and the Tdap antigens and the oligonucleotide are present in the immunogenic composition in amounts effective to stimulate an immune response against the Tdap antigens. In some embodiments, the immunogenic compositions further comprise an aluminum salt adjuvant to which the Tdap antigens are adsorbed.

The present disclosure also relates to methods for active booster immunization against tetanus, diphtheria, and pertussis, comprising: administering to a human subject an immunogenic composition comprising tetanus, diphtheria, and acellular pertussis (Tdap) antigens, an aluminum salt adjuvant to which the Tdap antigens are adsorbed, and a toll-like receptor 9 (TLR9) agonist, wherein the TLR9 agonist is an oligonucleotide of from 10 to 35 nucleotides in length comprising an unmethylated cytidine-phospho-guanosine (CpG) motif, the human subject is three years of age or older, the Tdap antigens comprise a tetanus toxoid (TT), a diphtheria toxoid (DT), a pertussis toxoid (PT), a pertussis filamentous haemagglutinin (FHA), and pertussis pertactin (PRN), and the Tdap antigens and the oligonucleotide are present in the immunogenic composition in amounts effective to stimulate an immune response against the Tdap antigens. The Tdap antigens are isolated or recombinant antigens, and do not comprise whole tetanus, diphtheria or pertussis bacterium. In some embodiments, the methods are effective in reducing pertussis colonization of nasal and/or pulmonary tissues of vaccinated subjects who have been exposed to pertussis.

A. Subjects

The intended recipients of the immunogenic compositions of the present disclosure are human subjects that are at least 3 years of age, and may have received at least two or three doses of a pediatric vaccine comprising diphtheria, tetanus, and acellular pertussis (DTaP) antigens, or diphtheria, tetanus, and whole cell pertussis (DTwP) antigens. In some embodiments, the human subjects are 4-6 years of age, and may have received a dose of a pediatric DTaP vaccine during their second year of life. In some embodiments, the human subjects are at least 7-10 years of age or older, and may have an incomplete or unknown pediatric vaccine history. In some embodiments, the human subjects are 11-18 years of age. In some embodiments, the human subjects are 19 years of age or older, 19-64 years of age, or 65 years of age or older. In some preferred embodiments, the human subject is at least 10 years of age, and has not received a dose of a pediatric vaccine or a booster vaccine within the previous 3, 4 or 5 years, wherein the pediatric vaccine comprises diphtheria, tetanus, and acellular pertussis (DTaP) antigens; or diphtheria, tetanus, and whole cell pertussis (DTwP) antigens; and the booster vaccine comprises tetanus and diphtheria toxoids (Td) antigens; or tetanus, diphtheria, and acellular pertussis (Tdap) antigens.

The immunogenic compositions of the present disclosure are expected to be suitable for preventing pertussis in newborns through maternal immunization. In particular, administration of the immunogenic composition to a pregnant woman in her first trimester of pregnancy is expected to result in the passive transfer of maternal antibodies to her infant. In this way, young infants may receive some degree of protection from pertussis before onset of a pediatric vaccine regimen. It may also be advantageous to administer the immunogenic compositions to healthcare workers, for both their own benefit and for decreasing risk of transmission of pertussis to individuals under their care.

B. Dose and Mode of Administration

The immunogenic compositions of the present disclosure are intended for active booster immunization against tetanus, diphtheria, and pertussis. The immunogenic compositions are to be administered by intramuscular injection as a single dose in a volume of about 0.5 mL (e.g., unit dose). In some embodiments, the intramuscular injection is into the deltoid muscle of the upper arm. The immunogenic compositions should not be administered intravenously, intradermally, or subcutaneously.

C. Methods For Stimulation of an Immune Response

The present disclosure relates to methods for stimulating an immune response to tetanus, diphtheria, and acellular pertussis (Tdap) antigens, comprising administering an immunogenic composition as described herein to a mammalian subject so as to stimulate an immune response against the Tdap antigens in the mammalian subject (e.g., human subject). In preferred embodiments, the immunogenic compositions are to be administered by intramuscular injection, optionally in a volume of about 0.5 mL (e.g., unit dose). In some embodiments, the intramuscular injection is into the deltoid muscle of the upper arm of a human subject in need thereof.

“Stimulating” an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating antibody production; stimulating interferon pathway-associated gene expression; stimulating chemoattractant-associated gene expression; and stimulating plasmacytoid dendritic cell maturation. In some preferred embodiments, stimulating an immune response comprises increasing an antigen-specific antibody response in the subject. Preferably, increasing the antigen-specific antibody response comprises increasing the concentration of antigen-specific antibodies at least 2, 3 or 4 fold higher than a pre-administration level at one month post administration and/or at least 1.5, 2.0, or 2.5 fold above the pre-administration level at six months post administration. Preferably, increasing the antigen-specific antibody response comprises increasing the concentration of antigen-specific antibodies at least 2, 3 or 4 fold above a threshold level (e.g., level of detection by ELISA in IU/ml) at one month post administration, and/or at least 1.5, 2.0, or 2.5 fold above the threshold level at six months post administration. Preferably, increasing the antigen-specific antibody response comprises increasing the concentration of antigen-specific antibodies above a minimum seroprotective level (e.g., for tetanus, at least 0.01 IU/ml as measured by a neutralization assay or at least 0.1 IU/ml as measured by ELISA).

### ENUMERATED EMBODIMENTS

1. A method for active booster immunization against tetanus, diphtheria, and pertussis, comprising:

administering to a human subject an immunogenic composition comprising tetanus, diphtheria, and acellular pertussis (Tdap) antigens, an aluminum salt adjuvant to which the Tdap antigens are adsorbed, and a toll-like receptor 9 (TLR9) agonist, wherein

the TLR9 agonist is an oligonucleotide of from 10 to 35 nucleotides in length comprising an unmethylated cytidine-phospho-guanosine (CpG) motif,

the human subject is three years of age or older,

the Tdap antigens comprise a tetanus toxoid (TT), a diphtheria toxoid (DT), a pertussis toxoid (PT), a pertussis filamentous haemagglutinin (FHA), and pertussis pertactin (PRN), and

the Tdap antigens and the oligonucleotide are present in the immunogenic composition in amounts effective to stimulate an immune response against the Tdap antigens.

2. The method of embodiment 1, wherein the oligonucleotide comprises the sequence 5′-AACGTTCGAG-3′ (SEQ ID NO:3).

3. The method of embodiment 1, wherein the oligonucleotide comprises the sequence 5′-TGACTGTGAA CGTTCGAGAT GA-3′(SEQ ID NO:1).

4. The method of any one of embodiments 1-3, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine.

5. The method of embodiment 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

6. The method of any one of embodiments 1-5, wherein the oligonucleotide comprises at least one phosphorothioate linkage, optionally wherein all nucleotide linkages are phosphorothioate linkages.

7. The method of any one of embodiments 1-6, wherein the oligonucleotide is a single-stranded oligodeoxynucleotide.

8. The method of any one of embodiments 1-7, wherein a 0.5 ml dose of the immunogenic composition comprises from about 375 μg to about 6000 μg of the oligonucleotide or from about 750 μg to about 3000 μg of the oligonucleotide, optionally wherein a 0.5 ml dose of the immunogenic composition comprises about 375 μg, about 750 μg, about 1500 μg, about 3000 μg, or 6000 μg about of the oligonucleotide.

9. The method of embodiment any one of embodiments 1-8, wherein the aluminum salt adjuvant comprises one or more of the group consisting of amorphous aluminum hydroxyphosphate sulfate, aluminum hydroxide, aluminum phosphate, and potassium aluminum sulfate.

10. The method of any one of embodiments 1-8, wherein the aluminum salt adjuvant comprises aluminum hydroxide.

11. The method of embodiment 9 or embodiment 10, wherein a 0.5 ml dose of the immunogenic composition comprises from about 0.25 to about 0.50 mg Al³⁺, optionally wherein a 0.5 ml dose of the immunogenic composition comprises from about 0.30 to about 0.40 mg Al³⁺.

12. The method of any one of embodiments 1-11, wherein a 0.5 ml dose of the immunogenic composition comprises about 5 Lf TT, about 2.0-2.5 Lf DT, about 2.5-8.0 μg PT, about 5.0-8.0 μg FHA, and about 2.5-3.0 μg PRN, optionally wherein a 0.5 ml dose of the immunogenic composition comprises about 5 Lf TT, about 2.5 Lf DT, about 8 μg PT, about 8 μg FHA, and about 2.5 μg PRN.

13. The method of any one of embodiments 1-12, wherein the immunogenic composition further comprises at least one additional antigen.

14. The method of embodiment 13, wherein the at least one additional antigen comprises one or both of a pertussis fimbriae (FIM) antigen and a pertussis adenylate cyclase (AC) antigen.

15. The method of embodiment 13 or embodiment 14, wherein the at least one additional antigen comprises an inactivated poliovirus, optionally wherein the inactivated poliovirus comprises one or more of a type 1 virus, a type 2 virus, and a type 3 virus.

16. The method of any one of embodiments 1-15, wherein the human subject is at least 10 years of age or older.

17. The method of any one of embodiments 1-16, wherein the human subject has not received a pediatric vaccine or a booster vaccine within the previous 3 years, wherein

the pediatric vaccine comprises diphtheria, tetanus, and acellular pertussis (DTaP) antigens; or diphtheria, tetanus, and whole cell pertussis (DTwP) antigens; and

the booster vaccine comprises tetanus and diphtheria (Td) antigens; or tetanus, diphtheria, and acellular pertussis (Tdap) antigens.

18. The method of embodiment 17, wherein the human subject is from 10-18 years of age.

19. The method of embodiment 17, wherein the human subject is 19 years of age or older, optionally wherein the human subject is from 19-64 years of age, or 65 years of age or older.

20. The method of any one of embodiments 1-19, wherein the Tdap antigens and the oligonucleotide are present in the immunogenic composition in amounts effective to stimulate an immune response against Tdap antigens by one month after administration, wherein the immune response comprises:

concentrations of anti-TT antibody, and anti-DT antibody of at least 0.4 international units/ml (IU/ml); and

concentrations of anti-PT antibody, anti-FHA antibody, and anti-PRN antibody of at least 2-fold higher in international units/ml (IU/ml) than pre-administration levels or limit of quantitation levels.

21. The method of embodiment 20, wherein the immune response comprises:

(i) one or both of: an anti-TT antibody concentration and an anti-DT antibody concentration of at least 1.0 IU/ml by one month post-administration; and/or

(ii) one or more of: an anti-PT antibody concentration, an anti-FHA antibody concentration, and an anti-PRN antibody concentration of at least 4-fold higher by one month post-administration, wherein the antibody concentrations are relative to either the pre-administration levels or limit of quantitation levels in international units/ml (IU/ml).

22. The method of any one of embodiments 20-21, wherein the immune response comprises a sustained tetanus and/or diphtheria immune response comprising:

one or both of an anti-TT antibody concentration and an anti-DT antibody concentration of at least 1.5, 2.0 or 2.5 times the pre-administration levels at about 6 months or about 12 months post-administration, optionally wherein one or both of the anti-TT antibody concentration and the anti-DT antibody concentration is at least 2, 3 or 4 times the pre-administration levels at about 6 months or about 12 months post-administration.

23. The method of any one of embodiments 20-22, wherein the immune response comprises a sustained pertussis immune response comprising:

one or more of an anti-PT antibody concentration, an anti-FHA antibody concentration, and an anti-PRN antibody concentration of at least 1.5, 2.0 or 2.5 times the pre-administration level at about 6 months or about 12 months post-administration, optionally one or more of the anti-PT antibody concentration, the anti-FHA antibody concentration, and the anti-PRN antibody concentration is at least 2, 3 or 4 times the pre-administration level at about 6 months or about 12 months post-administration.

24. The method of any one of embodiments 1-23, wherein the immunogenic composition has a satisfactory safety profile.

25. An immunogenic composition for active booster immunization against tetanus, diphtheria, and pertussis, comprising tetanus, diphtheria, and acellular pertussis (Tdap) antigens, an aluminum salt adjuvant to which the Tdap antigens are adsorbed, and a toll-like receptor 9 (TLR9) agonist, wherein

the TLR9 agonist is an oligonucleotide of from 10 to 35 nucleotides in length comprising an unmethylated cytidine-phospho-guanosine (CpG) motif,

the Tdap antigens comprise a tetanus toxoid (TT), a diphtheria toxoid (DT), a pertussis toxoid (PT), a pertussis filamentous haemagglutinin (FHA), and pertussis pertactin (PRN), and

the Tdap antigens and the oligonucleotide are present in the immunogenic composition in amounts effective to stimulate an immune response against the Tdap antigens in a human subject.

26. The composition of embodiment 25, wherein the oligonucleotide comprises the sequence 5′-AACGTTCGAG-3′ (SEQ ID NO:3).

27. The composition of embodiment 25, wherein the oligonucleotide comprises the sequence of 5′-TGACTGTGAA CGTTCGAGAT GA-3′(SEQ ID NO:1).

28. The composition of any one of embodiments 25-27, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine.

29. The composition of embodiment 28, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

30. The composition of any one of embodiments 25-29, wherein the oligonucleotide comprises at least one phosphorothioate linkage, optionally wherein all nucleotide linkages are phosphorothioate linkages.

31. The composition of any one of embodiments 25-30, wherein the oligonucleotide is a single-stranded oligodeoxynucleotide.

32. The composition of any one of embodiments 25-31, wherein a 0.5 ml dose of the immunogenic composition comprises from about 375 μg to about 6000 μg of the oligonucleotide or from about 750 μg to about 3000 μg of the oligonucleotide, optionally wherein a 0.5 ml dose of the immunogenic composition comprises about 375 μg, about 750 μg, about 1500 μg, about 3000 μg, or 6000 μg about of the oligonucleotide.

33. The composition of any one of embodiments 25-32, wherein the aluminum salt adjuvant comprises one or more of the group consisting of amorphous aluminum hydroxyphosphate sulfate, aluminum hydroxide, aluminum phosphate, and potassium aluminum sulfate.

34. The composition of any one of embodiments 25-32, wherein the aluminum salt adjuvant comprises aluminum hydroxide.

35. The composition of any one of embodiments 25-34, wherein a 0.5 ml dose of the immunogenic composition comprises from about 0.25 to about 0.50 mg Al³⁺, optionally wherein a 0.5 ml dose of the immunogenic composition comprises from about 0.30 to about 0.40 mg Al³⁺.

36. The composition of any one of embodiments 25-35, wherein a 0.5 ml dose of the immunogenic composition comprises about 5 Lf TT, about 2.0-2.5 Lf DT, about 2.5-8.0 μg PT, about 5.0-8.0 μg FHA, and about 2.5-3.0 μg PRN, optionally wherein a 0.5 ml dose of the immunogenic composition comprises about 5 Lf TT, about 2.5 Lf DT, about 8 μg PT, about 8 μg FHA, and about 2.5 μg PRN.

37. The composition of any one of embodiments 25-36, wherein the immunogenic composition further comprises at least one additional antigen.

38. The composition of embodiment 37, wherein the at least one additional antigen comprises one or both of a pertussis fimbriae (FIM) antigen and a pertussis adenylate cyclase (AC) antigen.

39. The composition of embodiment 37 or embodiment 38, wherein the at least one additional antigen comprises an inactivated poliovirus, optionally wherein the inactivated poliovirus comprises one or more of a type 1 virus, a type 2 virus, and a type 3 virus.

40. A kit comprising the composition of any one of embodiments 25-39, and instructions for administration of the composition to stimulate the immune response against the Tdap antigens in the human subject.

41. A kit comprising:

i) a first composition comprising tetanus, diphtheria, and acellular pertussis (Tdap) antigens and an aluminum salt adjuvant to which the Tdap antigens are adsorbed;

ii) a second composition comprising a TLR9 agonist; and

iii) instructions for mixing the first composition with the second composition to prepare an immunogenic composition, optionally wherein the immunogenic composition is the composition of any one of embodiments 25-39.

42. The kit of embodiment 41, further comprising:

iv) a further set of instructions for administration of the immunogenic composition to stimulate an immune response against the Tdap antigens in a human subject.

43. The kit of any one of embodiments 40-42, further comprising a syringe and needle for intramuscular injection of the immunogenic composition.

44. Use of the composition of any one of embodiments 25-39 for stimulating an immune response against tetanus, diphtheria, and acellular pertussis (Tdap) antigens in a human subject, the method comprising administering to the subject an effect amount of the composition.

45. Use of the composition of any one of embodiments 25-39 for protecting a human subject from infection with B. pertussis, the method comprising administering to the subject an effect amount of the composition.

46. Use of the composition of any one of embodiments 25-39 for preventing a human subject from contracting whooping cough, the method comprising administering to the subject an effect amount of the composition.

47. The use of any one of embodiments 44-46, wherein the composition is administered by intramuscular injection.

### EXAMPLES

Abbreviations: CpG (unmethylated cytidine-phospho-guanosine); DT (diphtheria toxoid); DTaP (diphtheria, tetanus, and acellular pertussis); DTwP (diphtheria, tetanus, and whole cell pertussis); FHA (filamentous haemagglutinin); GMC (geometric mean concentration); HD (human dose); Lf (flocculation unit); mcg (microgram); MVC (mock-vaccinated, challenged); NALT (nasal-associated lymphoid tissue); NVNC (non-vaccinated, non-challenged); Tdap (tetanus, reduced diphtheria, and acellular pertussis); IU/mL (international units/milliliter); OOR (outside of range); PRN (pertactin); PT (inactivated pertussis toxin); TLR9 (Toll-like receptor 9); TT (tetanus toxoid); and wP (whole cell pertussis).

Antigens: Tetanus, reduced diphtheria, and acellular pertussis (Tdap) antigens are produced independently during fermentation of four different bacterial strains: Clostridium tetani Harvard 49025 (tetanus toxin), Corynebacterium diphtheria PW8 (diphtheria toxin), Bordetella pertussis 165 (pertussis toxin), and Bordetella pertussis Tohama I (FHA and PRN). Fermenter harvest is collected and antigens are subsequently isolated, inactivated and sterile-filtered to produce individual antigens. For production of an alum-containing investigational vaccine, each antigen is further individually adsorbed on Al(OH)₃ before being blended into a Tdap-alum product.

Tetanus toxoid (TT) is produced by formalin detoxification of tetanus toxin produced by C. tetani strain Harvard 49205. Diphtheria toxoid (DT) is produced by formalin detoxification of diphtheria toxin produced by C. diphtheriae PW8 (CN2000). Pertussis toxoid (PT) is produced by formalin detoxification of pertussis toxin produced by B. pertussis 165. Filamentous haemagglutinin (FHA) is produced by formalin treatment of FHA produced by B. pertussis Tohama I. Pertactin (PRN) is produced by heat-inactivation of PRN-containing B. pertussis Tohama I cell mass.

### Example 1

### Immunogenicity of CpG Adjuvanted Tetanus Reduced Diphtheria Acellular Pertussis (Tdap) Vaccine in Mice

This example provides a description of a preclinical study conducted in Harlan mice to assess immunogenicity of two doses of various Tdap vaccines in homologous prime/boost regimens.

Vaccines. Four vaccines were tested, including Tdap alone (no adjuvant), Tdap-alum, Tdap-CpG, and Tdap-alum-CpG, each at two different antigen dilutions (undiluted and 1:4 dilution). The Tdap alone vaccine contained Tdap antigens but did not contain either alum or CpG. The Tdap-alum vaccine contained Tdap antigens adsorbed to 39 mcg Al(OH)₃ (aluminum hydroxide referred to herein as “alum”). The Tdap-CpG vaccine contained Tdap antigens mixed with 10 mcg of CpG 1018 (5′-TGACTGTGAA CGTTCGAGAT GA-3′, set forth as SEQ ID NO:1). The Tdap-alum-CpG vaccine contained Tdap antigens adsorbed to 39 mcg Al(OH)₃ and subsequently mixed with 10 mcg CpG 1018. Each 50 μl vaccine dose contained five different antigens as shown below in Table 1-1. For the 1:4 dilution groups, only the antigens were diluted, CpG 1018 and alum (where included) were dosed at 10 mcg and 39 mcg, respectively.

TABLE 1-1
Amount of Tdap Antigens in One Dose
of a Preclinical Tdap Vaccine

| Ag | TT | DT | PT | FHA | PRN |
|---|---|---|---|---|---|
| Dilution | (LF) | (LF) | (mcg) | (mcg) | (mcg) |
| None | 0.05 | 0.025 | 0.2 | 0.2 | 0.0625 |
| 1:4 | 0.0125 | 0.0063 | 0.05 | 0.05 | 0.0156 |

Mice were injected subcutaneously with 50 μl of a Tdap vaccine on day 0 and boosted on day 28. Blood was drawn on day 21 and day 42 for measurement of antibody levels by ELISA. There were 6-10 mice in each group. For each group, up to 5 serum sub-pools were made using serum from two mice each. Responses were measured and averaged for up to 4 sub-pools/group with the fifth sub-pool retained as a backup sample. Total average anti-PT, FHA, PRN, DT and TT IgG levels of sub-pooled serum from each group are shown below in Table 1-2. Individual mouse serum responses were also measured for mouse groups receiving non-diluted antigens. IgG levels are presented in IU/mL using assay standard curves constructed with reference sera.

TABLE 1-2
Total IgG Against Pertussis Antigens, DT, and TT in Pooled Serum

| | | Average Results (IU/mL) | | | | | | | | | |
|---|---|---|---|---|---|---|---|---|---|---|---|
| | Ag | 21 Days (after 1-Dose) | | | | | 42 Days (after 2-Doses) | | | | |
| Vaccine Group | Dilution | PT | FHA | PRN | DT | TT | PT | FHA | PRN | DT | TT |
| Tdap alone | None | OOR | OOR | OOR | OOR | OOR | 17 | 70 | 6 | OOR | 25 |
| Tdap-alum | | 1 | 30 | 1 | OOR | 4 | 18 | 162 | 8 | OOR | 92 |
| Tdap-CpG | | OOR | OOR | OOR | OOR | OOR | 1 | OOR | 1 | OOR | OOR |
| Tdap-alum-CpG | | 8 | 256 | 10 | OOR | 45 | 251 | 1496 | 79 | OOR | 458 |
| Tdap alone | 1:4 | OOR | OOR | OOR | OOR | OOR | OOR | OOR | OOR | OOR | OOR |
| Tdap-alum | | OOR | 15 | OOR | OOR | OOR | 21 | 52 | 3 | OOR | 72 |
| Tdap-CpG | | OOR | OOR | OOR | OOR | OOR | OOR | OOR | 1 | OOR | OOR |
| Tdap-alum-CpG | | OOR | 40 | 2 | OOR | 18 | 31 | 516 | 38 | OOR | 188 |

OOR = outside of range (below limitation of quantitation)

In the undiluted Tdap alone group, none of the six mice had a measurable antibody response at day 21 against any Tdap antigen. In the undiluted Tdap-alum group, five of ten mice had a measurable antibody response at day 21 against all three pertussis antigens (PT, FHA and PRN) and three of ten mice had a measurable antibody response at day 21 against TT. In contrast, in the undiluted Tdap-alum-CpG group, nine of ten mice had a measurable antibody response at day 21 against all three pertussis antigens (PT, FHA and PRN) and TT. Moreover the antibody response of the undiluted Tdap-alum-CpG group against each of the three pertussis antigens on day 21 (i.e. after a priming injection) was about 8-10-fold higher than the undiluted Tdap-alum group (i.e., Tdap vaccine lacking 1018 CpG) and was about 11-fold higher for TT antibody responses.

In the undiluted Tdap alone group, one of six mice had measurable antibody responses at day 42 against PT, FHA, PRN and TT, and because of this outlier, measurable antibody responses were observed at day 42 for this group (Table 1-2). In the undiluted Tdap-alum group, six of eight mice had measurable antibody responses at day 42 against PT, FHA, PRN and TT. Strikingly, in the undiluted Tdap-alum-CpG group, eight of eight mice had measurable antibody responses at day 42 against PT, FHA, PRN and TT. Moreover the antibody responses in the undiluted Tdap-alum-CpG group against each of the three pertussis antigens was over 9-fold higher on day 42 (i.e. after a booster injection) than the undiluted Tdap-alum group (i.e., Tdap vaccine lacking 1018 CpG). For both Tdap-alum and Tdap-alum-CpG groups, a clear booster effect of the second injection was observed for PT, FHA, PRN and TT (Table 1-2).

Antibody responses against DT were measured in sera of individual mice in the undiluted antigen groups in an endpoint titer assay because responses were not detected in the ELISA assay in comparison to reference sera (Table 1-2). No mice in either the Tdap alone group or the Tdap-alum group had a measurable antibody response against DT at day 21. By day 42, one of ten mice in the Tdap alone group and one of ten mice in the Tdap-alum group had a measurable antibody response against DT. In contrast, five of ten mice in the Tdap-alum-CpG group at day 21 (i.e. after a priming injection) and eight of eight mice in the Tdap-alum-CpG group at day 42 (i.e. after a booster injection) had a measurable antibody response against DT.

In summary, the Tdap-alum-CpG vaccine elicited higher antibody responses against Tdap antigens after one and two doses, than did the comparator vaccines.

### Example 2

### Th1/Th2/Th17 Balance in Pertussis Antigen Responses in Mice Subjected to Homologous or Heterologous Tdap Prime/Boost Regimens

This example provides a description of a preclinical study conducted in Harlan mice to assess immunogenicity of three doses of various Tdap vaccines in heterologous prime/boost regimens.

Vaccines. All alum formulations contained Tdap antigens adsorbed to either 9.8 or 39 mcg Al(OH)₃ (aluminum hydroxide referred to herein as “alum”). Four vaccine regimens were tested, each including one prime and 2 boosts: Tdap-alum (prime and 2 boosts); Tdap-alum prime+Tdap-CpG (2 boosts); Tdap alum prime+Tdap-alum-CpG (2 boosts); and Tdap-alum-CpG (prime and 2 boosts). The Tdap-alum vaccine contained Tdap antigens adsorbed to 39 mcg Al(OH)₃. The Tdap-CpG vaccine contained Tdap antigens mixed with 10 mcg of CpG 1018 (5′-TGACTGTGAA CGTTCGAGAT GA-3′, set forth as SEQ ID NO:1). The Tdap-alum-CpG vaccine contained Tdap antigens adsorbed to 39 mcg Al(OH)₃ and subsequently mixed with 10 mcg of CpG 1018. Each 50 μl vaccine dose contained five different antigens as shown below in Table 2-1. For the priming groups, antigens and alum were administered at 1:4 of the subsequent booster doses. CpG (where included) was dosed at 10 mcg in both priming and boosting doses.

TABLE 2-1
Amount of Tdap Antigens in One Dose of a Preclinical Tdap Vaccine

| | | | PT | FHA | PRN | Alum |
|---|---|---|---|---|---|---|
| Immunization | TT (LF) | DT (LF) | (mcg) | (mcg) | (mcg) | (mcg) |
| Prime | 0.0125 | 0.0063 | 0.05 | 0.05 | 0.0156 | 9.8 |
| Boosts | 0.05 | 0.025 | 0.2 | 0.2 | 0.0625 | 0 or 39 |

Mice were injected subcutaneously with 50 μl of a Tdap vaccine on day 0 and boosted on days 28 and 56 as shown below in Table 2-2. Blood was drawn on days 21, 42 and 68 for measurement of antibody levels by ELISA. There were 10 mice in each group. Sera was analyzed in pools of three (mice 1-3, 4-6 and 7-8) with samples from mice 9-10 kept as backups. Total IgG, as well as, IgG land IgG2a antigen-specific antibody responses were measured by ELISA with reference to a standard curve constructed using known quantities of IgG (IU/mL) This standard curve also served as a surrogate for quantifying IgG1 and IgG2a isotypes as specific standards for these were not available. Total average anti-PT, FHA, and PRN IgG results, as well as IgG1 and IgG2a levels, from each group sampled at day 68 (i.e. after priming and 2 booster doses) are shown below in Table 2-3. In vitro recall responses to Tdap antigen re-stimulation were measured at sacrifice (day 68) in supernatants of cultured spleen cells (96 hours) using the Luminex multi-analyte platform with reference to standard curves for IFN-γ, IL-5, and IL-17 all in pg/mL.

TABLE 2-2
Heterologous and Homologous Prime Boost Regimens

| | Prime^{b} | Bleed | Boost 1 | Bleed | Boost 2 | Bleed |
|---|---|---|---|---|---|---|
| Group^{a} | (Day 0) | Day | (Day 28) | Day | (Day 56) | Day^{c} |
| 1 | Tdap-alum | 21 | Tdap-alum | 42 | Tdap-alum | 68 |
| 2 | Tdap-alum | 21 | Tdap-CpG | 42 | Tdap-CpG | 68 |
| 3 | Tdap-alum | 21 | Tdap-alum- | 42 | Tdap-alum- | 68 |
| | | | CpG | | CpG | |
| 4 | Tdap-alum- | 21 | Tdap-alum- | 42 | Tdap-alum- | 68 |
| | CpG | | CpG | | CpG | |

^{a}N=10/group.
^{b}For priming groups, Tdap-alum vaccines were administered at a dose of 1:4 of the subsequent booster doses. The dilution was in saline so that less antigens and alum were present in the priming dose. CpG was always dosed at 10 mcg.
^{c}Terminal bleed for serum antibody titers, animal sacrifice and spleen harvest for antigen re-stimulation.

TABLE 2-3
Average Total IgG Levels and Estimated Average IgG1 and IgG2a Levels

| | | | | IgGl^{b} | IgG2a | IgG2a/IgG1 |
|---|---|---|---|---|---|---|
| Prime | Boost (2X) | Antigen | Total IgG^{a} | (Th2) | (Th1) | (Th1 Shift) |
| Tdap-alum | Tdap-alum | PT | 10 ± 2.7 | 12 ± 2.3 | 3.8 ± 3.1 | 0.3 |
| Tdap-alum | Tdap-CpG | | 10 ± 6.7 | 10 ± 6.3 | 30 ± 26 | 3.0 |
| Tdap-alum | T dap-alum-CpG | | 36 ± 13 | 33 ± 13 | 303 ± 111 | 9.2 |
| T dap-alum-CpG | T dap-alum-CpG | | 54 ± 4.9 | 35 ± 2.0 | 1018 ± 505 | 29 |
| Tdap-alum | Tdap-alum | FHA | 51 ± 12 | 82 ± 26 | 17 ± 12 | 0.2 |
| Tdap-alum | Tdap-CpG | | 147 ± 76 | 198 ± 91 | 232 ± 115 | 1.2 |
| Tdap-alum | T dap-alum-CpG | | 166 ± 34 | 124 ± 15 | 3452 ± 2594 | 28 |
| T dap-alum-CpG | T dap-alum-CpG | | 264 ± 7.3 | 72 ± 14 | 9776 ± 1497 | 136 |
| Tdap-alum | Tdap-alum | PRN | 13 ± 11 | 13 ± 11 | 13 ± 10 | 1.0 |
| Tdap-alum | Tdap-CpG | | 1.3 ± 0.4 | 1.0 ± 0.1 | 55 ± 52 | 55 |
| Tdap-alum | T dap-alum-CpG | | 34 ± 7.4 | 17 ± 2.2 | 970 ± 345 | 57 |
| T dap-alum-CpG | T dap-alum-CpG | | 14 + 8.5 | 4.6 ± 2.0 | 712 ± 489 | 155 |

^{a}Antibody levels in IU/mL, data presented as averages ± standard error of the mean for three sub-pools of mouse sera
^{b}IgGl and IgG2a levels are estimates based on comparison of assay readings to a total IgG standard curve.

The inclusion of CpG in either prime and boost or just boost vaccinations resulted in higher total IgG levels compared to Tdap-alum prime and boost groups. Homologous prime/boost regimens with Tdap-alum and Tdap-alum-CpG induced, respectively, Th2 versus Th1-biased antibody responses to pertussis antigens (PT, FHA, or PRN) as measured after two boosts. The IgG2a/IgG1 ratio (Th1/Th2 ratio) for the Tdap-alum prime and boost groups was 0.3 (for PT), 0.2 (for FHA), and 1.0 (for PRN). In contrast, Tdap-alum-CpG prime/boost strongly shifted the response to Th1, with IgG2a/IgG1 ratios of 29 (for PT), 136 (for FHA) and 155 (for PRN). The Tdap-alum/Tdap-CpG (no alum in boost) heterologous prime/boost regimen produced a Th1 shift as demonstrated by IgG2a/IgG1 ratios of 3.0 (for PT), 1.2 (for FHA), and 55 (for PRN). Heterologous prime/boost with Tdap-alum/Tdap-alum-CpG induced a more marked Th2 to Th1 shift with IgG2a/IgG1 ratios of 9.2 (for PT), 28 (for FHA), and 57 (for PRN).

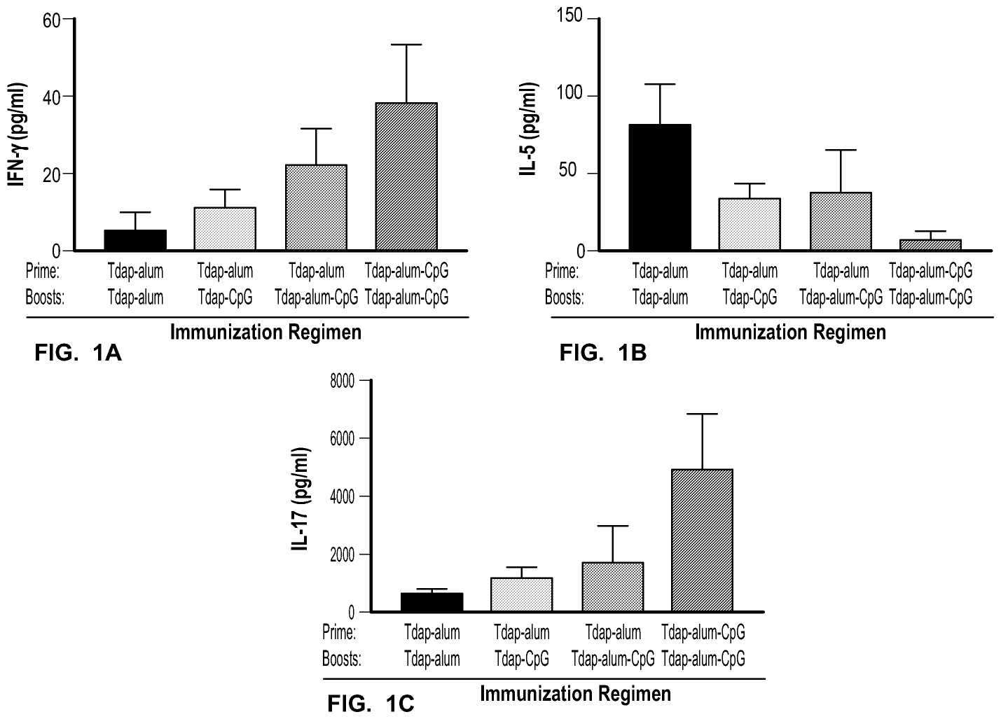

As shown in FIG. 1A-C, spleen cells from Tdap-alum prime/boosted mice re-stimulated with Tdap antigens produced IL-5 (Th2), but little IFN-γ (Th1) or IL-17 (Th17) after 96 hours of in vitro culture. In contrast, spleen cells from Tdap-alum-CpG prime boosted mice demonstrated a shift to Th1 (IFN-γ) and Th17 (IL-17) cytokine production upon in vitro re-stimulation. Boosting Tdap-alum primed mice twice with either Tdap-CpG or Tdap-alum-CpG demonstrated a shift to a more Th1/Th17 phenotype (lower IL-5, higher IFN-γ and IL-17) as compared to Tdap-alum prime and boosted mice. There was a trend to slightly more IFN-γ and IL-17 in Tdap-alum-CpG versus Tdap-CpG boosted mice, coinciding with the stronger shift to Th antibody responses in Tdap-alum-CpG boosted mice.

In summary, Tdap alum-primed mice boosted with Tdap-alum-CpG demonstrated higher levels of pertussis antigen-specific total IgG antibody responses compared to Tdap-alum prime/boosted controls. In addition, Tdap-alum-CpG boosted mice showed a marked shift to Th1 antibody responses (elevated IgG2a, associated with a higher IgG2a/IgG1 ratio) and increased production of IFN-γ and IL-17, and reduced IL-5 production, from spleen cells re-stimulated with Tdap antigens in vitro. The shift in Th2 to Th1 was also evident in Tdap-CpG (no alum) boosted mice.

### Example 3

### Efficacy of CpG Adjuvanted Tetanus Reduced Diphtheria Acellular Pertussis (Tdap) Vaccine in Mice

This example provides a description of a preclinical study conducted in CD1 mice to assess efficacy of two doses of various Tdap vaccines in homologous and heterologous prime/boost regimens. There were 14 groups in the study with 4-6 mice per treatment group, as listed in Table 3-1.

TABLE 3-1
Study Groups

| Group No. | Description |
|---|---|
| 1 | Control, naïve B. pertussis challenged = Mock Vaccinated |
| | Challenged (MVC) |
| 2 | SI DTwP 1/20 human dose (HD) |
| 3 | NIBSC wP 1/20 HD |
| 4 | Tdap 1/10 HD |
| 5 | Tdap 1/10 HD + 10 mcg 1018 |
| 6 | Tdap 1/20 HD |
| 7 | Tdap 1/20 HD + 10 mcg 1018 |
| 8 | Control, naïve non-infected = Non-Vaccinated, Non- |
| | Challenged (NVNC) |
| 9 | Control, naïve B. pertussis challenged = MVC |
| 10 | Tdap 1/40 HD |
| 11 | Tdap 1/40 HD + 10 mcg 1018 |
| 12 | Tdap 1/80 HD |
| 13 | Tdap 1/80 HD + 10 mcg 1018 |
| 14 | Tdap 1/20 HD (prime)/Tdap 1/20 HD + 10 mcg 1018 (boost) |

Study Design. Outbred, female CD1 mice were immunized at 5 weeks, boosted at 8 weeks, and challenged at 10 weeks. Mice were immunized by intramuscular injection of 50 μl of vaccine or saline control (0.9% NaCl). Mice were challenged by intranasal administration of about 26 million bacteria of B. pertussis, strain UT25 (PRN+): Groups 1-7 received 2.55×10⁷ per mouse (dose CFU/ml=1.28×10⁹); Groups 8-13 received 2.65×10⁷ per mouse (dose CFU/ml=1.33×10⁹); and Group 14 received 1.47×10⁷ per mouse (dose CFU/ml=7.33×10⁸). Bacteria were directly pipetted into the nares of anesthetized mice. Blood was drawn 2 or 3 days before challenge, and/or 3 days post-challenge.

Vaccines. Four vaccines were tested, including: Serum Institute DTwP (SI DTwP), National Institute for Biological Standards wP (NIBSC wP), Tdap-alum (referred to in Example 4 as Tdap), and Tdap-alum-CpG (referred to in Example 4 as Tdap CpG 1018). The whole cell pertussis vaccines (wP) were administered at 1/20 a human dose (HD), while the Tdap and Tdap-CpG 1018 vaccines were administered at 1/10, 1/20, 1/40 or 1/80 HD.

Efficacy. Efficacy of vaccines was assessed by measurement of various parameters of pertussis infection and pertussis-specific immunogenicity. Measurements included:

1. Bacterial burden in nasal-associated lymphoid tissue (NALT) of challenged mice;
2. Bacterial burden in nasal lavage of challenged mice;
3. Bacterial burden in lung and trachea of challenged mice;
4. Percentage change in bacterial burden due to inclusion of CpG 1018;
5. Lung weight;
6. Blood neutrophil counts;
7. IL-6 levels from lung homogenates;
8. IFN-γ levels from lung homogenates;
9. Pre-challenge and post-challenge serum IgG levels to PT, FHA, PRN, and whole bacteria; and
10. Phagocyte recruitment to the lungs.

TABLE 3-2
B. pertussis Burden as Average CFU/ml

| | Nasal- | | |
|---|---|---|---|
| | Associated | | |
| | Lymphoid | Nasal | Lung + |
| Group | Tissue | Wash | Trachea |
| MVC | 21650 | 25700 | 3912222 |
| Tdap 1/10 | 1275 | 1821 | 138508 |
| Tdap 1/10 + 1018 | 745 | 725 | 492 |
| Tdap 1/20 | 1055 | 4256 | 155750 |
| Tdap 1/20 + 1018 | 370 | 5066 | 1878 |
| NVNC | ND | ND | ND |
| Tdap 1/40 | 1446 | 2540 | 353110 |
| Tdap 1/40 + 1018 | 105 | 1251 | 21850 |
| Tdap 1/80 | 1456 | 1273 | 555600 |
| Tdap 1/80 + 1018 | 494 | 900 | 87310 |
| Tdap 1/20/Tdap 1/20 + 1018 | 708 | 3021 | 4792 |
| SI DTwP | 385 | 1321 | 9558 |
| NIBSC wP | 370 | 971 | 11506 |

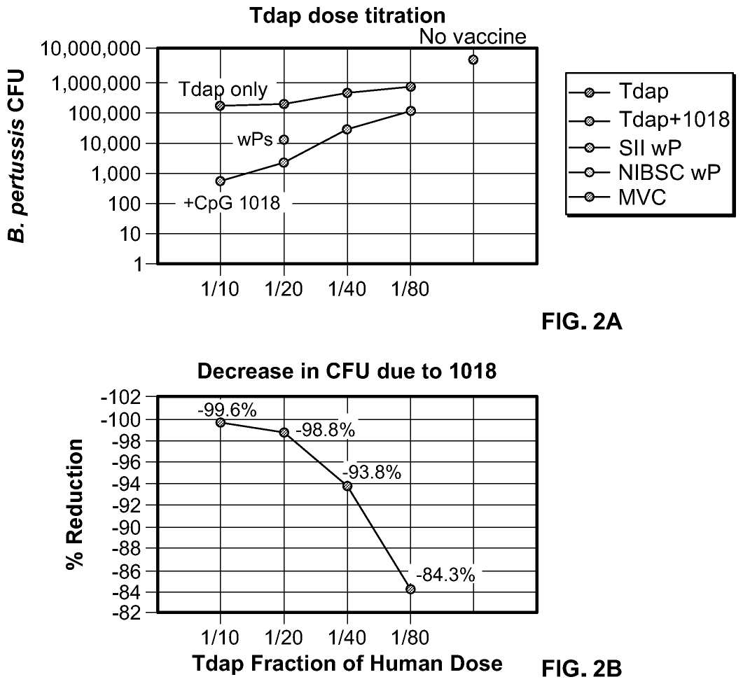

Bacterial burden in NALT, nasal wash (1 ml PBS flush), and lung+trachea was measured three days post B. pertussis challenge. As shown in Table 3-2 and FIG. 2A, inclusion of CpG 1018 in Tdap vaccines reduced the bacterial burden in nasal and pulmonary tissues of B. pertussis-challenged recipients, at all four of the Tdap antigen concentrations tested. Remarkably, inclusion of CpG 1018 reduced CFU in lungs+trachea by 99.6% (1/10), 98.8% (1/20), 93.8% (1/40) and 84.3% (1/80), respectively (FIG. 2B). In fact, the CpG 1018-containing vaccines including 1/10 or 1/20 HD Tdap had bacterial burdens comparable to recipients of the two wP-containing vaccines. The heterologous Tdap 1/20/Tdap 1/20+1018 immunization regimen produced post challenge bacterial burden data intermediate between homologous Tdap 1/20 and Tdap 1/20+1018 regimens.

Lungs were weighed three days post B. pertussis challenge before homogenization as a way to assess pulmonary inflammation. Lungs of mice of the mock vaccinated, and the two wP vaccinated groups weighed substantially more than did the lungs of mice of the non-vaccinated, non-challenged (NCNV) group, and the nine Tdap-vaccinated groups regardless of the inclusion of CpG 1018. These results indicate that the inclusion of CpG 1018 does not cause high levels of inflammation in order to reduce bacterial burden.

Neutrophil counts in whole blood were measured three days post B. pertussis challenge. Neutrophil counts of mice of the mock vaccinated, and the two wP vaccinated groups were considerably higher than the neutrophil counts of mice of the non-vaccinated, non-challenged (NCNV) group. Neutrophil counts of mice of the nine Tdap-vaccinated groups were lower than neutrophil counts of the two wP vaccinated groups, with somewhat higher counts observed in recipients of the CpG 1018-containing Tdap vaccines.

IL-6 and IFN-γ levels of lung homogenates were measured three days post B. pertussis challenge. IL-6 levels of mice of the nine Tdap-vaccinated groups were lower than IL-6 levels of the two wP vaccinated groups. A 84-95% reduction in IL-6 levels were observed when comparing IL-6 levels of recipients of the CpG 1018-containing Tdap vaccines to recipients of the SII DTwP vaccine. IFN-γ levels of mice of the Tdap-vaccinated groups were lower than IFN-γ levels of the two wP vaccinated groups. However, IFN-γ levels were higher in recipients of the CpG 1018-containing Tdap vaccines than in recipients of Tdap vaccines containing equivalent antigen doses in the absence of CpG 1018 suggestive of increased Th1 response in response to the CpG 1018-adjuvanted Tdap vaccines.

TABLE 3-3
Pre-challenge IgG Antibody Titers

| Group | PT | FHA | PRN | B. pertussis |
|---|---|---|---|---|
| MVC | 50 | 50 | 50 | 50 |
| Tdap 1/10 | 40960 | 46080 | 20850 | 28160 |
| Tdap 1/10 + 1018 | 53760 | 163840 | 52780 | 76800 |
| Tdap 1/20 | 10880 | 29440 | 6460 | 16640 |
| Tdap 1/20 + 1018 | 58880 | 158720 | 7760 | 87040 |
| NVNC | 50 | 50 | 50 | 50 |
| Tdap 1/40 | 5760 | 69120 | 80 | 23040 |
| Tdap 1/40 + 1018 | 10320 | 115200 | 120 | 51200 |
| Tdap 1/80 | 1940 | 24320 | 50 | 27520 |
| Tdap 1/80 + 1018 | 4813 | 43520 | 70 | 25920 |
| SI DTwP | 3230 | 1075 | 4000 | 89600 |
| NIBSC wP | 50 | 14720 | 3750 | 76800 |

TABLE 3-4
Post-challenge IgG Antibody Titers

| Group | PT | FHA | PRN | B. pertussis |
|---|---|---|---|---|
| MVC | 50 | 50 | 50 | 50 |
| Tdap 1/10 | 25600 | 44800 | 15760 | 72533 |
| Tdap 1/10 + 1018 | 20480 | 122880 | 74260 | 97280 |
| Tdap 1/20 | 3520 | 24320 | 6500 | 40960 |
| Tdap 1/20 + 1018 | 24320 | 102400 | 18560 | 133120 |
| NVNC | 50 | 50 | 50 | 50 |
| Tdap 1/40 | 6400 | 53760 | 130 | 40960 |
| Tdap 1/40 + 1018 | 7760 | 166400 | 200 | 84480 |
| Tdap 1/80 | 2600 | 27520 | 138 | 21760 |
| Tdap 1/80 + 1018 | 4850 | 67200 | 88 | 38400 |
| Tdap 1/20/Tdap 1/20 + 1018 | 17920 | 51200 | 22490 | 64000 |
| SI DTwP | 1630 | 890 | 5440 | 76800 |
| NIBSC wP | 50 | 17920 | 5960 | 51200 |

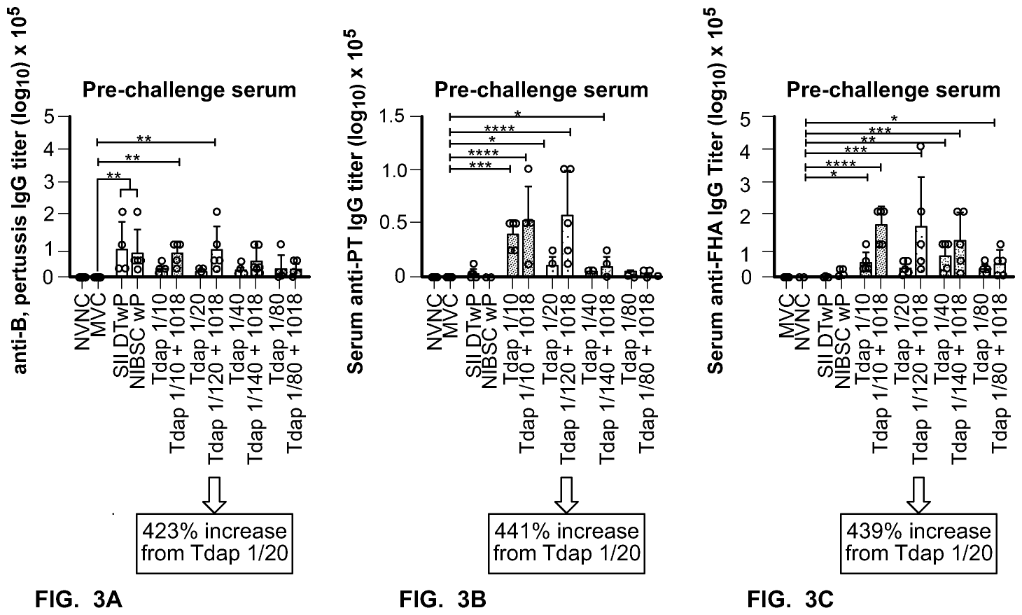

Pertussis-specific serum IgG antibody titers were measured by ELISA from blood drawn 2 or 3 days before challenge (Table 3-3), and/or 3 days post-challenge (Table 3-4). In general, recipients of the CpG 1018-containing Tdap vaccines had higher pre- and post-challenge, serum IgG titers to PT, FHA, PRN and whole bacteria than did recipients of Tdap vaccines containing equivalent antigen doses in the absence of CpG 1018. The positive affect of CpG 1018 on antibody titers was most pronounced in the groups receiving higher antigen doses (Tdap 1/10 and 1/20 HD) as shown in FIG. 3A-C.

Differential cellular counts in lung homogenates were measured three days post B. pertussis challenge. Lung neutrophil, dendritic cell, macrophage and monocyte counts of mice of the mock vaccinated, and the two wP vaccinated groups were considerably higher than the counts for these cell populations in mice of the non-vaccinated, non-challenged (NCNV) group. Cell counts of mice of the nine Tdap-vaccinated groups trended lower than cell counts of the two wP vaccinated groups, with somewhat higher counts observed in several groups of the CpG 1018-containing Tdap vaccinated animals.

### Example 4

### Immunogenicity of CpG Adjuvanted Tetanus Reduced Diphtheria Acellular Pertussis (Tdap) Vaccine in Humans

This example provides a description of a clinical study to be conducted in healthy subjects to assess safety, tolerability and immunogenicity of an investigational Tdap vaccine in comparison to an FDA-approved Tdap vaccine.

Vaccines. Tdap-alum-1018 is an investigational vaccine being developed by Dynavax Technologies Corporation (Emeryville, Calif.). Each 0.5 mL dose of the investigational vaccine contains Tdap antigens (5 Lf of TT, 2.5 Lf of DT, 8 mcg of PT, 8 mcg of FHA and 2.5 mcg of PRN), 0.39 mg alum, and either 1500 mcg or 3000 mcg of CpG 1018. The Tdap-alum-1018 vaccine is prepared just prior to use by mixing Tdap antigens adsorbed onto aluminum hydroxide (not more than 0.39 mg Al³⁺) with CpG 1018 (5′-TGACTGTGAA CGTTCGAGAT GA-3′, set forth as SEQ ID NO:1). BOOSTRIX® (tetanus toxoid, reduced diphtheria toxoid and acellular pertussis) is an FDA-approved vaccine marketed by GlaxoSmithKline (Research Triangle Park, N.C.). Each 0.5 mL dose of BOOSTRIX® contains Tdap antigens (5 Lf of TT, 2.5 Lf of DT, 8 mcg of PT, 8 mcg of FHA and 2.5 mcg of PRN) individually adsorbed onto aluminum hydroxide (not more than 0.39 mg Al³⁺), 4.5 mg sodium chloride, ≤100 mcg residual formaldehyde, and ≤100 mcg polysorbate 80. Placebo is preservative free, 0.9% Sodium Chloride Injection, USP (sterile isotonic saline).

Objectives: 1) to assess the safety and tolerability of Tdap-alum-1018; 2) to assess the immunogenicity to acellular pertussis antigens of Tdap-alum-1018 compared with BOOSTRIX® at 1 month (week 4) after a single booster vaccination; and 3) to assess the immunogenicity to tetanus and diphtheria of Tdap-alum-1018 compared with BOOSTRIX® at 1 month (week 4) after a single booster vaccination. A further objective is 4) to assess the duration of the antibody response against pertussis of Tdap-alum-1018 compared with BOOSTRIX® at 6 and 12 months after a single booster vaccination.

Safety and tolerability is assessed by monitoring local and systemic post-injection reactions, clinical and laboratory adverse events, and serious adverse events Immunogenicity to vaccine antigens is assessed by measurements made from samples obtained from study subjects at Baseline (Day 1) and at Week 4 (Day 29±5). Additional immunogenicity assessments are made at one or more of Week 8, Week 12, Month 6 (weeks 24-26), and Month 12 (weeks 48-52). Levels of antibodies to tetanus, diphtheria, and pertussis antigens in serum are determined by ELISA. T and B cell responses to pertussis antigens of PBMC are also measured. Additionally, levels of pertussis toxin-neutralizing antibodies, opsonizing antibodies, and other functional antibody responses in serum or plasma are measured. Nasal swabs obtained from study subjects are subjected to microRNA testing to assess B. pertussis colonization.

Immunogenicity to pertussis antigens is assessed by separately measuring antibody response rates against PT, FHA and PRN. A PT, FHA and PRN antibody response are each defined as one of the following: 1) a measurable post-vaccination antibody concentration in initially seronegative subjects, such as post-vaccination antibody concentrations greater than or equal to 2, 3 or 4 times a threshold level (e.g., limit of detection); 2) an increase of at least 2, 3 or 4 times the pre-vaccination antibody concentration in initially seropositive subjects.

Immunogenicity to pertussis antigens is also assessed by determining the geometric mean concentration (GMC) for each of anti-PT, anti-FHA, and anti-PRN antibodies. Immunogenicity to tetanus and diphtheria antigens is assessed by separately measuring antibody response rates against TT and DT. A TT and DT antibody response are each defined as one of the following: 1) in subjects with pre-vaccination antibody levels <0.1 IU/mL, post-vaccination antibody levels ≥0.4 IU/mL; and 2) in subjects with pre-vaccination antibody level ≥0.1 IU/mL, an increase of at least 4 times the pre-vaccination level. Duration of the antibody response against pertussis is measured by measuring antibody titers for PT, FHA and PRN.

Study Design. The study is conducted as a two part randomized, open-label, study of Tdap booster vaccines in healthy subjects 10-22 years of age. Part 1 is conducted in healthy adult subjects 18-22 years of age. Part 2 is conducted in healthy adolescent subjects 10-17 years of age. After screening, subjects are randomized into the following treatment groups: Treatment A: Tdap-alum-1018 (dose 1 or dose 2); and Treatment B: BOOSTRIX®. Subjects will have a baseline blood draw before receiving the randomized treatment vaccine at Day 1 to determine pre-vaccination antibody levels. Vaccines are administered as a single 0.5-mL intramuscular injection into the deltoid muscle. Blood draws for measurement of post-vaccination antibody levels are done at week 4 (˜Day 29), and optionally at 12, 24 and 52 weeks, or at 4, 8 and 12 weeks.

Adult participants receive 1 dose of their assigned treatment (Tdap-1018 1500 mcg, Tdap-1018 3000 mcg, or BOOSTRIX®) on Day 1, and 1 dose (Tdap-1018 1500 mcg, Tdap-1018 3000 mcg, or placebo) at Week 8. Adolescent participants receive 1 dose of their assigned treatment (Tdap-1018 1500 mcg, Tdap-1018 3000 mcg, or BOOSTRIX®) on Day 1. In adults, blood draws for measuring post-vaccination antibody levels are done at Weeks 4, 8, and 12 and exploratory evaluations are done at Weeks 4 and 12. In adolescents, blood draws for measuring post-vaccination antibody levels are done at Week 4. Safety assessments are done at Weeks 4, 12, and 20 in adults and at Weeks 4 and 12 in adolescents.

Study Population. Inclusion and exclusion criteria for study subjects include but are not limited to the listing provided below. Inclusion criteria include all of: male or female, 18-22 years of age (Part 1) or 10-17 years of age (Part 2); have documentation of all previous pertussis immunizations (e.g., 2 or more prior acellular pertussis vaccinations); in good health based on medical history, physical exam, and laboratory evaluation; and human immunodeficiency virus-seronegative. Exclusion criteria include any one of the following: received an acellular Tdap booster within the previous 3 years; history of diphtheria, tetanus or pertussis disease; history of autoimmune disease; and/or has a condition, therapy or laboratory abnormality that may confound the results of the study. Potentially confounding therapies include receiving any of the following: any inactivated virus vaccine within 21 days of study vaccine injection; any live virus vaccine, systemic corticosteroids or other immune suppressive medication, granulocyte or granulocyte-macrophage colony-stimulating factor, or other investigational medicinal agent within 28 days of study vaccine injection; immunoglobulins or any blood products within 3 months of study vaccine injection; or injection of deoxyribonucleic acid plasmids or oligonucleotides at any time.

Results. Recipients of Tdap-alum-1018 are contemplated to have higher concentrations of antibodies to one or more of PT, FHA and PRN antigens at four weeks post booster vaccination than recipients of BOOSTRIX®. Likewise, recipients of Tdap-alum-1018 are contemplated to have higher concentrations of antibodies to one or both of TT and DT antigens at four weeks post booster vaccination than recipients of BOOSTRIX®. Additionally, a higher percentage of subjects receiving Tdap-alum-1018 are contemplated to have anti-TT antibodies ≥0.1 IU/mL or ≥1.0 IU/mL, and/or anti-DT antibodies ≥0.1 IU/mL or ≥1.0 IU/mL.

### Example 5

Efficacy of a CpG Adjuvanted Tetanus Reduced Diphtheria Acellular Pertussis (Tdap) Vaccine for Reduction in Nasal Colonization in B. pertussis-Challenged Humans

This example provides a description of a clinical study to be conducted in healthy subjects to assess safety, tolerability, immunogenicity and protective effects of an investigational Tdap vaccine in comparison to an FDA-approved Tdap vaccine. In particular, human subjects are to receive a controlled intranasal challenge with wild-type B. pertussis after they have received, zero, one or two doses of a Tdap vaccine. Challenged subjects are treated with azithromycin treatment to eradicate bacterial colonization as described (de Graaf et al., Clinical Infectious Diseases, 71(2): 403-411, 2020).

Vaccines. Tdap-alum-1018 is an investigational vaccine being developed by Dynavax Technologies Corporation (Emeryville, Calif.). Each 0.5 mL dose of the investigational vaccine contains Tdap antigens (5 Lf of TT, 2.5 Lf of DT, 8 mcg of PT, 8 mcg of FHA and 2.5 mcg of PRN), 0.39 mg alum, and either 1500 mcg or 3000 mcg of CpG 1018. The Tdap-alum-1018 vaccine is prepared just prior to use by mixing Tdap antigens adsorbed onto aluminum hydroxide (not more than 0.39 mg Al³⁺) with CpG 1018 (5′-TGACTGTGAA CGTTCGAGAT GA-3′, set forth as SEQ ID NO:1). BOOSTRIX® (tetanus toxoid, reduced diphtheria toxoid and acellular pertussis) is an FDA-approved vaccine marketed by GlaxoSmithKline (Research Triangle Park, N.C.). Each 0.5 mL dose of BOOSTRIX® contains Tdap antigens (5 Lf of TT, 2.5 Lf of DT, 8 mcg of PT, 8 mcg of FHA and 2.5 mcg of PRN) individually adsorbed onto aluminum hydroxide (not more than 0.39 mg Al³⁺), 4.5 mg sodium chloride, ≤100 mcg residual formaldehyde, and ≤100 mcg polysorbate 80.

Objectives: 1) To assess the efficacy of Tdap-alum-1018 for reduction in nasal colonization post challenge (lower bacterial titers in nasal washes and/or more rapid clearance of bacteria) in comparison to the acellular vaccine; 2) To assess protection against early clinical symptoms in challenged subjects; 3) To assess safety and tolerability of Tdap-alum-1018; and 4) To assess the immunogenicity to acellular pertussis antigens of Tdap-alum-1018 compared with BOOSTRIX® at week 4 after a single or at week 8 after two vaccinations pre-B. pertussis intranasal challenge.

Safety and tolerability are assessed by monitoring local and systemic post-injection reactions, clinical and laboratory adverse events, and serious adverse events Immunogenicity to vaccine antigens is assessed by measurements made from samples obtained from study subjects at Baseline (Day 1), at Week 4 (Day 29±5)/pre-challenge or, in subjects receiving 2 doses, at Week 8 (Day 60±5)/pre-challenge Immune responses are also assessed at 2 weeks post challenge. Levels of antibodies to tetanus, diphtheria, and pertussis antigens in serum are determined by ELISA. T cell responses to pertussis antigens of PBMC are also measured. Additionally, levels of pertussis toxin-neutralizing antibodies, opsonizing antibodies, and other functional antibody responses in serum are measured.

Immunogenicity to pertussis antigens is assessed by separately measuring antibody response rates against PT, FHA and PRN. A PT, FHA and PRN antibody response are each defined as one of the following: 1) a measurable post-vaccination antibody concentration in initially seronegative subjects, such as post-vaccination antibody concentrations greater than or equal to 2, 3 or 4 times a threshold level (e.g., limit of detection); 2) an increase of at least 2, 3 or 4 times the pre-vaccination antibody concentration in initially seropositive subjects.

Immunogenicity to pertussis antigens is also assessed by determining the geometric mean concentration (GMC) for each of anti-PT, anti-FHA, and anti-PRN antibodies. Immunogenicity to tetanus and diphtheria antigens is assessed by separately measuring antibody response rates against TT and DT. A TT and DT antibody response are each defined as one of the following: 1) in subjects with pre-vaccination antibody levels <0.1 IU/mL, post-vaccination antibody levels ≥0.4 IU/mL; and 2) in subjects with pre-vaccination antibody level ≥0.1 IU/mL, an increase of at least 4 times the pre-vaccination level.

Immunogenicity to pertussis antigens is further assessed by antibody characterization assays and evaluation of functional antibody responses. Functional antibody responses are assessed by serum-based assays to measure antibody neutralization of pertussis toxin and to measure antibody opsonization activity in an opsonophagocytosis assay. In addition, antibody isotyping, Fc receptor-binding, and additional antibody functional readouts are assessed in a Systems Serology analysis.

Moreover, immunogenicity to pertussis antigens is also assessed by measuring antigen-recall responses in T cells. PBMCs from subjects are re-stimulated in vitro with pertussis antigens (PT, FHA, and PRN) and intracellular cytokine expression is measured by flow cytometry.

Study Design. The study is conducted as a randomized, open-label, study on the effect of Tdap booster vaccines on healthy subjects' responses to B. pertussis intranasal challenge. Healthy adult subjects are either acellular-primed (18-22 years of age) or whole cell vaccine primed (23-50 years of age), but not boosted within the previous 3 years, and are negative for serum antibody responses to pertussis toxin. After screening, subjects are randomized into the following treatment groups (n=50/group): 1) No vaccine; 2) BOOSTRIX®; 3) In subjects who previously received acellular or whole cell vaccine, one dose of Tdap-alum-1018 with 1500 or 3000 mcg of 1018; and 4) In subjects who previously received acellular vaccine, two doses of Tdap-alum-1018 with 1500 or 3000 mcg of 1018. Subjects have a baseline blood draw before receiving the treatment vaccine at Day 1 to determine pre-vaccination antibody levels and baseline functional antibody response and T cell responses (PBMCs). Vaccines are administered as a single 0.5-mL intramuscular injection into the deltoid muscle. Blood draws for measurement of post-vaccination antibody levels, functional antibody responses and PBMC for T cell responses are done at week 4 (˜Day 29 in subjects receiving a single pre-challenge vaccine dose) or week 8 (˜Day 60 in subjects receiving 2 pre-challenge vaccine doses). After blood draws, subjects are inoculated intransally with B. pertussis strain D420 (approximately 10⁵ colony forming units). Blood draws are also done at week 2 post-B. pertussis challenge to assess potential differential effects of pertussis colonization on immune responses in the different study groups. Safety, colonization, and bacterial shedding are monitored over 21 days in an inpatient facility. Colonization is assessed by culture and quantitative polymerase chain reaction. Azithromycin is administered from Day 21 to clear nasal colonization by B. pertussis.

Study Population. Inclusion and exclusion criteria for study subjects include but are not limited to the listing provided below. Inclusion criteria include all of: male or female, 18-50 years of age; have documentation of all previous pertussis immunizations (e.g., 2 or more prior acellular or whole cell pertussis vaccinations); in good health based on medical history, physical exam, and laboratory evaluation; and human immunodeficiency virus-seronegative. Exclusion criteria include any one of the following: received an acellular Tdap booster within the previous 3 years; history of diphtheria, tetanus or pertussis disease; history of autoimmune disease; and/or has a condition, therapy or laboratory abnormality that may confound the results of the study. Potentially confounding therapies include receiving any of the following: any inactivated virus vaccine within 21 days of study vaccine injection; any live virus vaccine, systemic corticosteroids or other immune suppressive medication, granulocyte or granulocyte-macrophage colony-stimulating factor, or other investigational medicinal agent within 28 days of study vaccine injection; immunoglobulins or any blood products within 3 months of study vaccine injection; or injection of deoxyribonucleic acid plasmids or oligonucleotides at any time.

Results. Recipients of Tdap-alum-1018 are contemplated to have lower bacterial burden and/or faster clearance of bacteria post intranasal inoculation, as well as less and/or milder disease-associated symptoms. The Tdap-alum-1018 vaccinated subjects are contemplated to demonstrate higher concentrations of antibodies to one or more of PT, FHA and PRN antigens at four or eight weeks post booster vaccination than recipients of BOOSTRIX®. Likewise, recipients of Tdap-alum-1018 are contemplated to have higher concentrations of antibodies to one or both of TT and DT antigens at four or eight weeks post booster vaccination than recipients of BOOSTRIX®. Additionally, a higher percentage of subjects receiving Tdap-alum-1018 are contemplated to have anti-TT antibodies ≥0.1 IU/mL or ≥1.0 IU/mL, and/or anti-DT antibodies ≥0.1 IU/mL or ≥1.0 IU/mL. Anticipated effects of Tdap-alum-1018 versus BOOSTRIX® vaccination also include differential antibody isotyping and Fc receptor-binding profiles, as well as increased functional antibody responses. Tdap-alum-1018 versus BOOSTRIX® vaccination are contemplated to have differential effects on T cell cytokine expression profiles, with Tdap-alum-1018 vaccinated subjects expected to demonstrate more Th1- and Th17 biased responses (IFN-γ, IL-17 expression) and BOOSTRIX® vaccinated subjects will be expected to demonstrate more Th2-biased responses (IL-4, IL-5, or IL-13).

Although the foregoing disclosure has been described in some detail by way of illustration and example for purposes of clarity and understanding, it will be apparent to those skilled in the art that certain changes and modifications may be practiced. Therefore, the examples should not be construed as limiting the scope of the disclosure, which is delineated by the appended claims.

We claim:

1. A method for active booster immunization against tetanus, diphtheria, and pertussis, comprising:
administering to a human subject an immunogenic composition comprising tetanus, diphtheria, and acellular pertussis (Tdap) antigens, an aluminum salt adjuvant to which the Tdap antigens are adsorbed, and a toll-like receptor 9 (TLR9) agonist, wherein
the TLR9 agonist is an oligonucleotide of from 10 to 35 nucleotides in length comprising an unmethylated cytidine-phospho-guanosine (CpG) motif,
the human subject is three years of age or older,
the Tdap antigens comprise a tetanus toxoid (TT), a diphtheria toxoid (DT), a pertussis toxoid (PT), a pertussis filamentous haemagglutinin (FHA), and pertussis pertactin (PRN), and
the Tdap antigens and the oligonucleotide are present in the immunogenic composition in amounts effective to stimulate an immune response against the Tdap antigens.

2. The method of claim 1, wherein the oligonucleotide comprises the sequence 5′-AACGTTCGAG-3′ (SEQ ID NO:3).

3. The method of claim 1, wherein the oligonucleotide comprises the sequence 5′-TGACTGTGAA CGTTCGAGAT GA-3′(SEQ ID NO:1).

4. The method of claim 1, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine.

5. The method of claim 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

6. The method of claim 3, wherein the oligonucleotide comprises at least one phosphorothioate linkage, or wherein all nucleotide linkages are phosphorothioate linkages.

7. The method of claim 6, wherein the oligonucleotide is a single-stranded oligodeoxynucleotide.

8. The method of claim 6, wherein a 0.5 ml dose of the immunogenic composition comprises from about 375 μg to about 6000 μg of the oligonucleotide or from about 750 μg to about 3000 μg of the oligonucleotide, or wherein a 0.5 ml dose of the immunogenic composition comprises about 375 μg, about 750 μg, about 1500 μg, about 3000 μg, or 6000 μg about of the oligonucleotide.

9. The method of claim 8, wherein the aluminum salt adjuvant comprises one or more of the group consisting of amorphous aluminum hydroxyphosphate sulfate, aluminum hydroxide, aluminum phosphate, and potassium aluminum sulfate.

10. The method of claim 8, wherein the aluminum salt adjuvant comprises aluminum hydroxide.

11. The method of claim 9, wherein a 0.5 ml dose of the immunogenic composition comprises from about 0.25 to about 0.50 mg Al³⁺, or wherein a 0.5 ml dose of the immunogenic composition comprises from about 0.30 to about 0.40 mg Al³⁺.

12. The method of claim 11, wherein a 0.5 ml dose of the immunogenic composition comprises about 5 Lf TT, about 2.5 Lf DT, about 8 μg PT, about 8 μg FHA, and about 2.5 μg PRN.

13. The method of claim 12, wherein the immunogenic composition further comprises at least one additional antigen.

14. The method of claim 13, wherein the at least one additional antigen comprises one or both of a pertussis fimbriae (FIM) antigen and a pertussis adenylate cyclase (AC) antigen.

15. The method of claim 13, wherein the at least one additional antigen comprises an inactivated poliovirus, optionally wherein the inactivated poliovirus comprises one or more of a type 1 virus, a type 2 virus, and a type 3 virus.

16. The method of any one of claims 1-15, wherein the human subject is at least 10 years of age or older.

17. The method of claim 16, wherein the human subject has not received a pediatric vaccine or a booster vaccine within the previous 3 years, wherein
the pediatric vaccine comprises diphtheria, tetanus, and acellular pertussis (DTaP) antigens; or diphtheria, tetanus, and whole cell pertussis (DTwP) antigens; and
the booster vaccine comprises tetanus and diphtheria (Td) antigens; or tetanus, diphtheria, and acellular pertussis (Tdap) antigens.

18. The method of claim 17, wherein the human subject is from 10-18 years of age.

19. The method of claim 17, wherein the human subject is 19 years of age or older, or wherein the human subject is from 19-64 years of age, or 65 years of age or older.

20. The method of claim 17, wherein the Tdap antigens and the oligonucleotide are present in the immunogenic composition in amounts effective to stimulate an immune response against Tdap antigens by one month after administration, wherein the immune response comprises:
concentrations of anti-TT antibody, and anti-DT antibody of at least 0.4 international units/ml (IU/ml); and
concentrations of anti-PT antibody, anti-FHA antibody, and anti-PRN antibody of at least 2-fold higher in international units/ml (IU/ml) than pre-administration levels or limit of quantitation levels.

21. The method of claim 20, wherein the immune response comprises:
(i) one or both of: an anti-TT antibody concentration and an anti-DT antibody concentration of at least 1.0 IU/ml by one month post-administration; and/or
(ii) one or more of: an anti-PT antibody concentration, an anti-FHA antibody concentration, and an anti-PRN antibody concentration of at least 4-fold higher by one month post-administration, wherein the antibody concentrations are relative to either the pre-administration levels or limit of quantitation levels in international units/ml (IU/ml).

22. The method of claim 20, wherein the immune response comprises a sustained tetanus and/or diphtheria immune response comprising:
one or both of an anti-TT antibody concentration and an anti-DT antibody concentration of at least 1.5, 2.0 or 2.5 times the pre-administration levels at about 6 months or about 12 months post-administration, or wherein one or both of the anti-TT antibody concentration and the anti-DT antibody concentration is at least 2, 3 or 4 times the pre-administration levels at about 6 months or about 12 months post-administration.

23. The method of claim 20, wherein the immune response comprises a sustained pertussis immune response comprising:
one or more of an anti-PT antibody concentration, an anti-FHA antibody concentration, and an anti-PRN antibody concentration of at least 1.5, 2.0 or 2.5 times the pre-administration level at about 6 months or about 12 months post-administration, or wherein one or more of the anti-PT antibody concentration, the anti-FHA antibody concentration, and the anti-PRN antibody concentration is at least 2, 3 or 4 times the pre-administration level at about 6 months or about 12 months post-administration.

24. The method of claim 20, wherein the immunogenic composition has a satisfactory safety profile.

25. An immunogenic composition for active booster immunization against tetanus, diphtheria, and pertussis, comprising tetanus, diphtheria, and acellular pertussis (Tdap) antigens, an aluminum salt adjuvant to which the Tdap antigens are adsorbed, and a toll-like receptor 9 (TLR9) agonist, wherein
the TLR9 agonist is an oligonucleotide of from 10 to 35 nucleotides in length comprising an unmethylated cytidine-phospho-guanosine (CpG) motif,
the Tdap antigens comprise a tetanus toxoid (TT), a diphtheria toxoid (DT), a pertussis toxoid (PT), a pertussis filamentous haemagglutinin (FHA), and pertussis pertactin (PRN), and
the Tdap antigens and the oligonucleotide are present in the immunogenic composition in amounts effective to stimulate an immune response against the Tdap antigens in a human subject.

26. The composition of claim 25, wherein the oligonucleotide comprises the sequence 5′-AACGTTCGAG-3′ (SEQ ID NO:3).

27. The composition of claim 25, wherein the oligonucleotide comprises the sequence of 5′-TGACTGTGAA CGTTCGAGAT GA-3′(SEQ ID NO:1).

28. The composition of claim 25, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine.

29. The composition of claim 28, wherein the oligonucleotide comprises the sequence 5′-TCG₁AACG₁TTCG₁-3′ (SEQ ID NO:2) in which G₁ is 2′-deoxy-7-deazaguanosine, or wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

30. The composition of claim 27, wherein the oligonucleotide comprises at least one phosphorothioate linkage, or wherein all nucleotide linkages are phosphorothioate linkages.

31. The composition of claim 30, wherein the oligonucleotide is a single-stranded oligodeoxynucleotide.

32. The composition of claim 30, wherein a 0.5 ml dose of the immunogenic composition comprises from about 375 μg to about 6000 μg of the oligonucleotide or from about 750 μg to about 3000 μg of the oligonucleotide, or wherein a 0.5 ml dose of the immunogenic composition comprises about 375 μg, about 750 μg, about 1500 μg, about 3000 μg, or 6000 μg about of the oligonucleotide.

33. The composition of claim 32, wherein the aluminum salt adjuvant comprises one or more of the group consisting of amorphous aluminum hydroxyphosphate sulfate, aluminum hydroxide, aluminum phosphate, and potassium aluminum sulfate.

34. The composition of claim 32, wherein the aluminum salt adjuvant comprises aluminum hydroxide.

35. The composition of claim 32, wherein a 0.5 ml dose of the immunogenic composition comprises from about 0.25 to about 0.50 mg Al³⁺, or wherein a 0.5 ml dose of the immunogenic composition comprises from about 0.30 to about 0.40 mg Al³⁺.

36. The composition of claim 35, wherein a 0.5 ml dose of the immunogenic composition comprises about 5 Lf TT, about 2.5 Lf DT, about 8 μg PT, about 8 μg FHA, and about 2.5 μg PRN.

37. The composition of claim 36, wherein the immunogenic composition further comprises at least one additional antigen.

38. The composition of claim 37, wherein the at least one additional antigen comprises one or both of a pertussis fimbriae (FIM) antigen and a pertussis adenylate cyclase (AC) antigen.

39. The composition of claim 37, wherein the at least one additional antigen comprises an inactivated poliovirus, optionally wherein the inactivated poliovirus comprises one or more of a type 1 virus, a type 2 virus, and a type 3 virus.

40. A kit comprising the composition of any one of claims 25-39, and instructions for administration of the composition to stimulate the immune response against the Tdap antigens in the human subject.

41. A kit comprising:
i) a first composition comprising tetanus, diphtheria, and acellular pertussis (Tdap) antigens and an aluminum salt adjuvant to which the Tdap antigens are adsorbed;
ii) a second composition comprising a TLR9 agonist; and
iii) instructions for mixing the first composition with the second composition to prepare the immunogenic composition of any one of claims 25-39.

42. The kit of claim 41, further comprising:
iv) a further set of instructions for administration of the immunogenic composition to stimulate an immune response against the Tdap antigens in a human subject.

43. The kit of claim 42, further comprising a syringe and needle for intramuscular injection of the immunogenic composition.
